(19) United States
(12) Patent Application Publication
(10) Pub. No.: US 2007/0004979 A1
(43) Pub. Date: Jan. 4, 2007

(54) Clip and method for epicardial placement of temporary heart pacing electrodes

(76) Inventors: Alex Wojciechowicz, Princeton, NJ (US); Carol Wojciechowicz, Princeton, NJ (US); Peter I. Praeger, Briarcliff Manor, NY (US)

Correspondence Address: Richard C Woodbridge;Synnestvedt Lechner & Woodbridge, PO Box 592, Princeton, NJ, 08542-0592, US

(21) Appl. No.: 10/484,186

(22) PCT Filed: Jul. 16, 2002
(86) PCT No.: PCT/US02/22662

§ 371 Date: Aug. 5, 2006

(30) Foreign Application Priority Data
Jul. 16, 2001 (US) 60305690

(51) Int. Cl. A61B 5/04

(52) U.S. Cl. 600/386

(57) ABSTRACT

The present invention provides for a sutureless clip with an electrode releasably attached to a spring clip electrode holder for use in placement of electrodes for heart pacemakers, the crimping tool for attaching the clips to the heart and the method of attachment. The preferred electrode holder is in the form of a spring clip formed by bending around one end of the clip to contact either the inside or outside surface of the clip. An electrode lead wire is releaseably held against the sutureless clip by the spring clip and can be removed by pulling on the wire, leaving the sutureless clip in place. A specialized crimping tool and it's method of use is disclosed for attaching the clip to the heart.

### CROSS REFERENCE TO RELATED APPLICATIONS

This Application claims the benefit of U.S. Provisional Patent Application Ser. No. 60/305,690 filed on Jul. 16, 2001 entitled “Clip and Method for Epicardial Placement of Temporary Heart Placement” by Alex Wojciechowicz and Peter I. Praeger, the entire contents and substance of which are hereby incorporated by reference.

### BACKGROUND OF THE INVENTION

1. Field of the Invention

The invention relates to a small metal clip with an electrode holder that is used to releasably attach an electrode to a heart for temporary heart pacing and the method of installing the clip.

2. Description of Related Art

Following many heart surgeries, it is necessary to connect a temporary pacing device to the heart before the patient's chest is closed. This connection is made with a wire, one end of which is connected to the heart and the other connected to an external pacing device. The pacing may be either unipolar, requiring a single electrode or bipolar, requiring dual electrodes.

Pacing electrodes may be sutured onto the right atrium or ventricular wall or as in U.S. Pat. No. 5,782,901 to Praeger, the electrodes may be held in place by a clip, which holds the electrode in place. In Praeger, a small generally V shaped clip has an electrode holder on the outer surface. The electrode is friction fit into a generally V shaped groove machined on the outer surface of the clip. The clip is crimped on to the outside of the heart prior to closing the chest cavity. The clip is designed to be held in place with minimum compression of the heart muscle in order to prevent atrophication of the tissue, which allows the clip to remain in place permanently. The electrode is run out through the chest cavity and is connected to the pacing device. When heart pacing is no longer required, the electrode may be removed by tugging the exposed end with sufficient force to release the opposite end from the friction fit. This offers advantages over suturing since the chest cavity need not be reopened to remove the electrode. The problem lies in fabricating the clip such that the pull out force required to remove the electrode from the clip is reproducibly in the required range. It is difficult to manufacture a clip with a groove, which will hold an electrode with the 0.5 to 2.0 pounds of pull out force. Machining grooves into clips that are on the order of 2 to 6 millimeters with a thickness of 0.2 to 0.5 millimeters such that electrodes friction fit in the grooves will have a reproducible pull out force has proven to be difficult in practice.

Clips have been proposed with alternate configurations for holding the electrode on the clip. Such a method is illustrated in U.S. Pat. No. 6,256,543 B1 to Spence. Spence discloses a temporary pacemaker lead which is connected to the heart and has an electrode with a wire loop on its end. The loop engages a scorpion type pincer or a friction fit with a loop on the clip. Other clips have been proposed for holding electrodes on the exterior of the heart (See U.S. Pat. Nos. 4,144,890 and 4,177,818) but caused much greater traumatic injury during installation due to prongs which had to be embedded into the heart.

The present invention offers the advantages of little or no traumatic injury to the external heart wall, ease of installation with a specially designed crimping tool and ease of removal of the electrode wire. A further advantage is that the end of the electrode wire does not require a loop which could snag and cause injury when being extracted.

### SUMMARY OF THE INVENTION

Briefly described, the invention comprises a small metal clip with an electrode holder that is used to releasably attach an electrode to a heart for temporary heart pacing. The clip is fabricated from a small strip of metal which is bent into a U, V or C shape. One end of the strip has an electrode holder attached. The electrode holder is designed to clasp a small electrode used for connecting a temporary heart pacemaker. The electrode is held by the holder with a force sufficient to keep it in electrical contact with the heart but may be released from the clip by tugging on the exposed end. The electrode holder may be in the form of a spring clip or clamp on the outside of the clip. The spring clip may be formed as an integral part of the clip forming a bend at one end of the flat plate around either the inside or the outside of the clip, so that the end goes completely around and rests on the surface of the plate. The electrode is then placed held against the plate. The release force required to pull out the electrode can be controlled by the spring tension in the spring clip. Additional bends may be formed in the plate to form the desired profile. If the electrode holding spring clip is on the inside of the clip, a hole through the clip may be provided to allow the electrode to pass through the clip and be held by the spring clip. Alternatively, the electrode can be held to the clip by a metal plate or cap welded to the outside of the clip. The clip is held in place by crimping it directly to the external heart wall.

An alternative embodiment of the clip has the electrode lead wire held in place on the clip by a cap that is welded on to the outside of the clip. The release force in this case is controlled by the initial force applied to the cap before welding.

The clip is installed during surgery prior to closing the patient's chest cavity. A single clip may be installed for unipolar applications or multiple clips may be installed such as for bipolar applications. The electrode lead is coiled or folded in a crimping tool and then the clip is placed in the form fitting jaws. The clip is placed on the heart wall and the crimping tool is used to crimp the clip on to the heart. The crimping tool is removed from the clip and the electrode is uncoiled from the crimping tool as the tool is removed. The clip remains in place permanently. The electrode is lead through the chest wall and remains exposed for connection to an external heart-pacing device. The electrode may be removed by applying tension to the electrode sufficient to pull it out of the spring clip. Typically this is between ½ to 1 pound of force. The clip remains inside the chest connected to the heart.

The present invention offers the advantages of little or no traumatic injury to the external heart wall, ease of installation with a specially designed crimping tool and each of removal of the electrode wire. A further advantage is that the end of the electrode wire does not require a loop which could snag and cause injury when being extracted.

The invention may be more fully understood by reference to the following drawings.

### BRIEF DESCRIPTION OF THE DRAWINGS

FIG. 1 shows a side cross sectional view of a clip with spring electrode holder on the outside of the clip.

FIG. 2 shows a front perspective view of a clip with spring electrode holder on the outside of the clip.

FIG. 3 shows an top plan view of a sutureless clip with spring electrode holder on the inside of the clip.

FIG. 4 shows a side cross sectional view of a sutureless clip with spring electrode holder on the inside of the clip.

FIG. 5 shows a perspective underside view of a sutureless clip with spring electrode holder on the inside of the clip.

FIGS. 6A and 6B show a crimping tool.

FIGS. 7A, 7B and 7C show a crimping tool with electrode storage tubes.

FIGS. 8A-8D show the steps required for installation of the sutureless clip using a crimping tool.

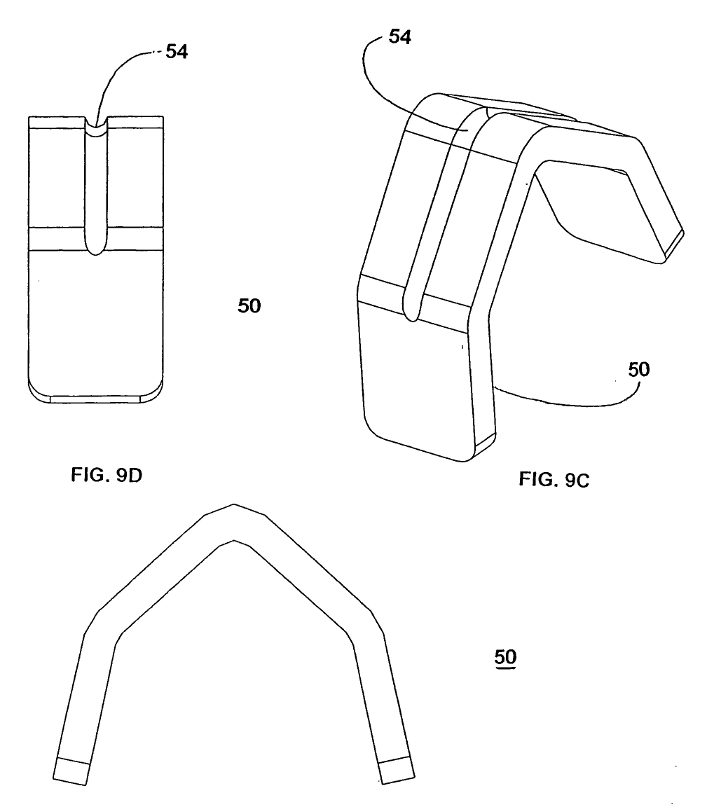

FIGS. 9A-9E show an alternative embodiment of the invention which includes a welded cap on the outside of the clip.

### DETAILED DESCRIPTION OF THE PREFERRED EMBODIMENT

During the course of this description, like numbers will be used to identify like elements according to the different views which illustrate the invention.

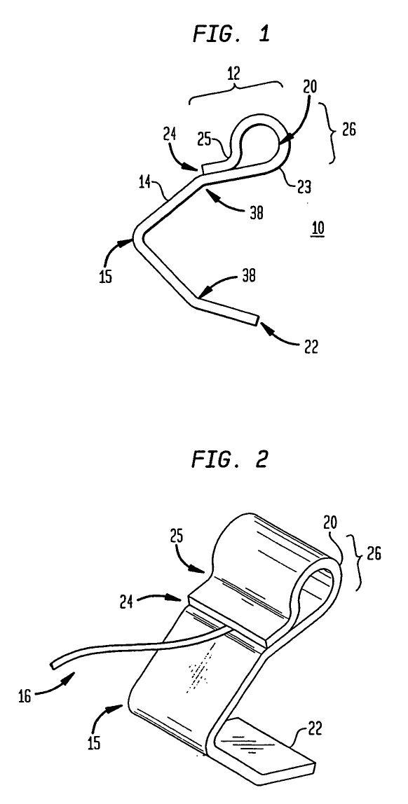

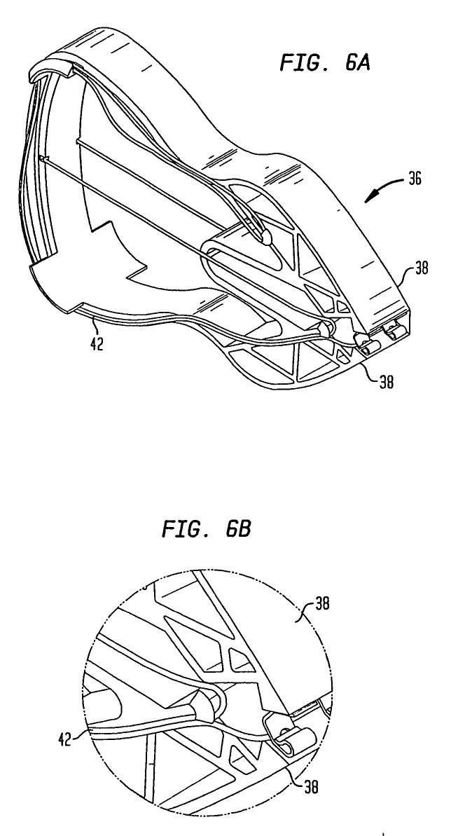

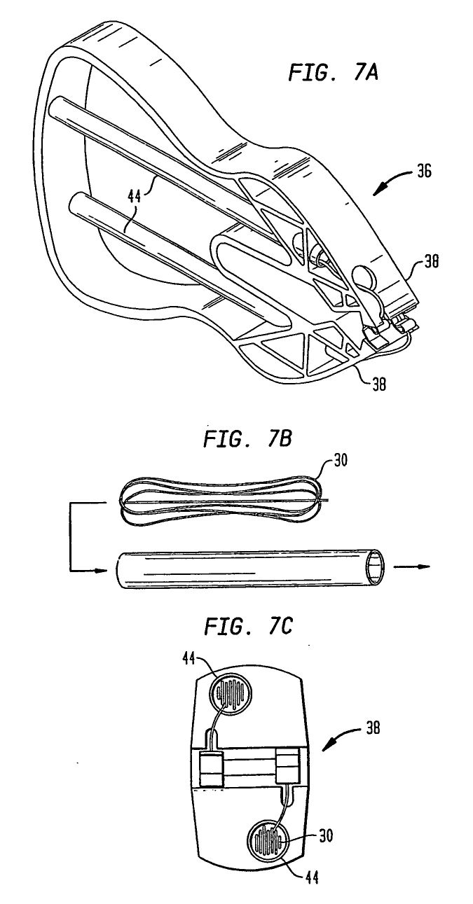

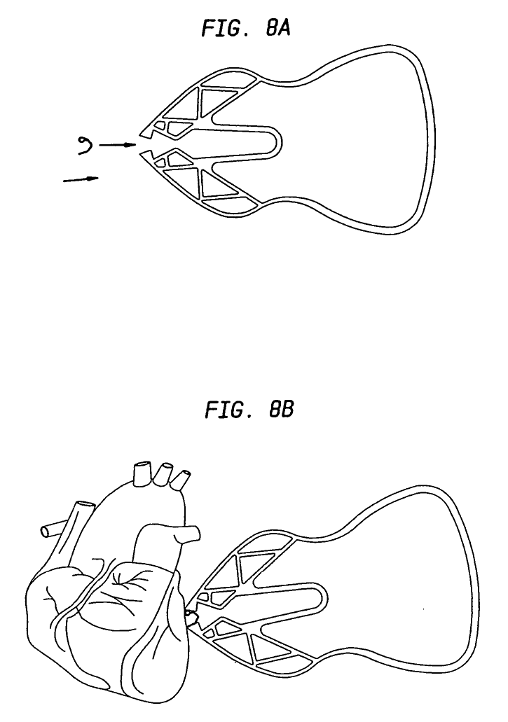

FIGS. 1-5 show a small clip that is used as an electrode for temporary heart pacing when crimped to the surface tissue of a heart chamber. The clip has a modified V shaped cross section with a major bend 15 and additional bends 38 and an integral round spring coil 26, 28 that can be located adjacent to either the outside 26 or inside 28 surface of the clip 10. The clip 10 is attached to the surface tissue by inserting the tissue between the open jaws 22, 23 of the clip. A special crimping tool 36 is then used to close the jaws 22, 23 together so that tissue is held firmly only along the front edge of the clip haws. A special feature of the clip 10 and crimping tool 36 is that tissue trapped between the front edge and back of the clip is compressed only enough for electrical conduction to the tissue but not enough to stop blood flow. This is important because this clip will remain on the heart after the pacing lead is removed and will prevent the entrapped tissue from atrophying.

The spring clip 10 is formed by bending one end of a small metal plate 24 around to contact either the inside or outside surface of the plate. The formation of the spring clip, namely the radius of the bend 20, 21, the thickness of the plate, and the material used in the fabrication of the plate, are all factors which determine the force holding the electrode lead wire. A second small radius bend 25, 27 is included to provide a contact area for the electrode lead wire. If the spring clip is on the inside of the clip, the electrode lead wire may be inserted into the spring clip by running the electrode lead wire around the outside of the clip and around to the inside surface, or the electrode lead wire may be run through a small hole 31 in the clip to the inside of the clip and then inserted into the spring clip 28.

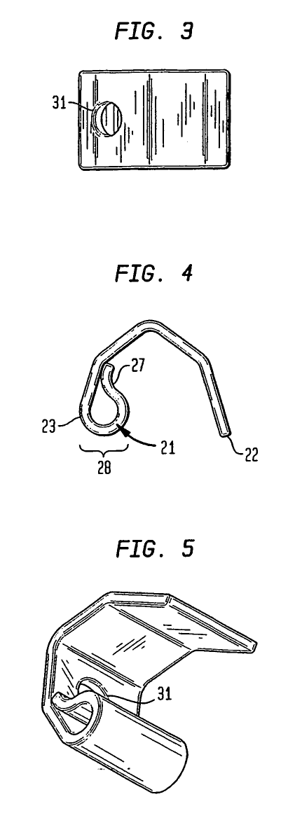

An insulated electrode lead wire 30 conducts pacing current from the pacemaker located outside the body to the clip electrode 10 attached to the heart surface. It is attached to the electrode during manufacture by passing the wire conductor under the lip of the spring coil as shown in FIG. 2. It is important that this wire be attached to the electrode secure enough that it doesn't pull free from the electrode due to the gyrations of the beating heart, yet will release fairly easily when the surgeon decides to remove the lead using gentle pulling tension. Experimentation has shown that a removal force of between ½ to 2 pounds is desirable and sufficient for this application. The lead removal force is dependent on how tightly the spring holds the lead wire in place. Obviously, the coil geometry, material, and material thickness needed to produce the required holding force can be theoretically or empirically determined.

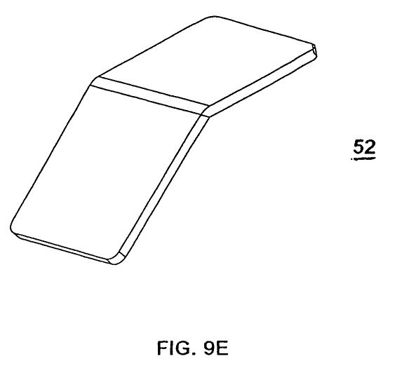

An alternative embodiment of the clip 50 shown in FIG. 9A-9E has the electrode lead wire held in place on the clip 50 by a cap 52 that is welded on to the outside of the clip. The release force in this case is controlled by the initial force applied to the cap before welding. A recess 54 may be machined into the top surface of the clip to hold the electrode lead wire in place prior to the cap being welded on

The typical size of the clip can vary, but generally will be in the range of 2 to 6 millimeters in both width and height with a material thickness of 0.2 to 0.5 millimeters. Acceptable materials for the clip are well known to those skilled in the art of heart pacing. A partial list of suitable materials is 316L stainless steel, F-67 titanium, platinum, iridium, Elgiloy, pyrolitic carbon coating, ipanium, graphite platinum and iridium oxide. Special coatings on the inside conducting surface such as iridium oxide, carbon, or titanium nitrate could be applied to obtain optimum pacing characteristics. The clip surface not touching the heart could also be electrically insulated with a biocompatible coating such as aprylene or silicone to further enhance performance.

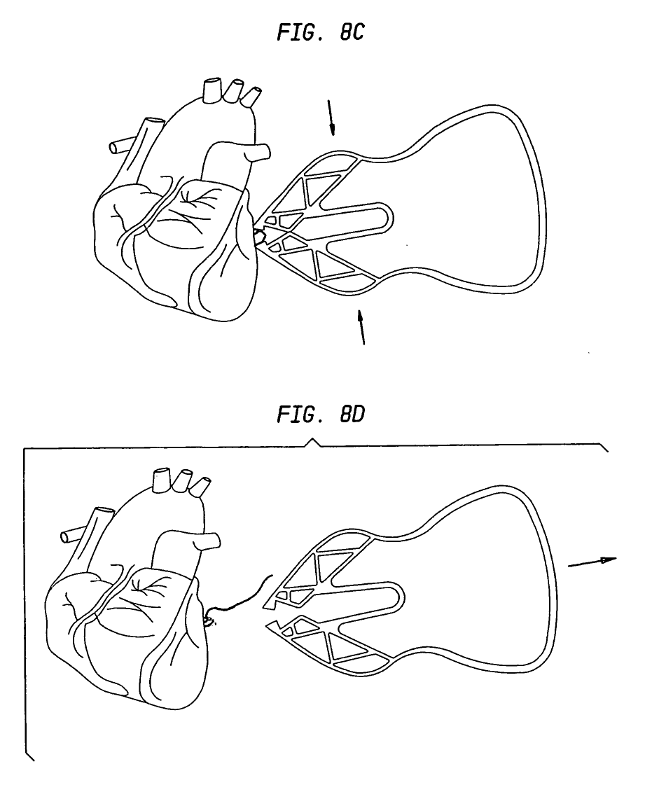

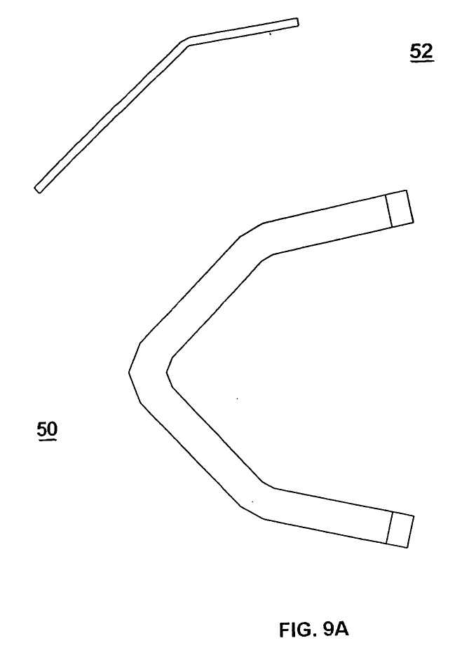

Temporary pacing leads are almost always used in pairs attached to the same heart chamber. One lead 30 is used for sensing and the other used for pacing. This type of pacing is called bipolar pacing. Most experts recommend for bipolar pacing that spacing of the electrode pair be approximately 1 centimeter. This spacing may be adjusted as required to achieve the optimum. A special crimping tool 36 and technique allows the surgeon to apply both electrodes (shown in FIGS. 6A, 6B and 7A, 7B) simultaneously and precisely at the optimum spacing. The crimping tool 36 is also used to store the wire leads 30 and chest-piercing needle while in the package and during installation of the electrodes. The jaws 38 of the crimping tool have special cavities that hold the electrodes securely in place with optimum spacing. In the preferred embodiment, the wire leads 30 and chest-piercing needle are coiled an inserted into a tube molded into the crimping tool where they remain until the electrodes are crimped into place. The wire lead and needle are then pulled out of the tube.

There are occasions when only a single pacing electrode is used in conjunction with a skin ground lead. The crimping tool may be used to store an install only one electrode 30.

There are several advantages of the present design over the prior art. The first is a reproducible release force when removing the lead wire from the sutureless clip. The second is ease of manufacturing and finally positive electrical contact between the clip and living tissue can be maintained.

While the invention has been described with reference to the preferred embodiment thereof, it will be appreciated by those of ordinary skill in the art that modifications can be made to the structure and elements of the invention without departing from the spirit and scope of the invention as a whole.

We claim:

1. A sutureless clip apparatus for attaching an electrode to living tissue comprising:
a flat strip having a first end and a second end and, a top side and a bottom side with a first bend on said bottom side between said first and second ends so as to bring said first end closer to said second end; and
an electrode holder attached to one of said first or second ends for releasably holding said electrode,
wherein said clip is attachable to living tissue with a crimping tool and said electrode is releasably held by said electrode holder.

2. The apparatus of claim 1 wherein said flat strip is made of a physiologically acceptable material selected from the group consisting of: 316L stainless steel, F-67 titanium, platinum, iridium, Elgiloy, pyrolitic carbon coating ipanium, graphite platinum and iridium oxide.

3. The apparatus of claim 2 wherein said flat strip is bent into a V shape.

4. The apparatus of claim 3 wherein said flat strip is bent into a modified V shaped cross section with additional bends located between said first bend and said first end and said first bend and said second end.

5. The apparatus of claim 4 wherein the force required to release said electrode from said electrode holder is between 0.5 and 2 pounds.

6. The apparatus of claim 5 wherein said electrode holder comprises a spring clip and includes:
a bend on said top side of said flat strip such that said first or second end is bent around to make contact with said top side and releasably hold said electrode.

7. The apparatus of claim 5 wherein said electrode holder comprises a spring clip and includes:
a bend on said bottom side of said flat strip such that said first or second end is bent around to make contact with said bottom side and releasably hold said electrode.

8. The apparatus of claim 7 wherein said flat strip has a through aperture to accept said electrode, said aperture being located on the same end of said flat strip as said spring clip.

9. The apparatus of claim 5 wherein said electrode holder comprises a flat cap welded on said top side of said sutureless clip to hold said electrode lead wire in place.

10. A crimping tool apparatus having a first half and a second half connected by a hinge means for crimping a sutureless clip to living tissue, said tool further comprising:
a first end comprising jaws capable of holding at least two of said sutureless clips and having a predetermined separation of said clips, and a second end having a biasing means for applying a bias force to said sutureless clips to hold said clips in said jaws prior to crimping, wherein when said first ends are forced together, said halves pivot about said hinge and said jaws crimp said sutureless clip on to living tissue.

11. The apparatus of claim 9 wherein said jaws have a profile which matches the outer surface of said clip.

12. The apparatus of claim 10 wherein said hinge is a living hinge.

13. The device of claim 11 further including an electrode guide means located on said first end of said crimping tool to guide an electrode lead wire after said clip is attached to living tissue.

14. The device of claim 12 further including at least one through aperture on said hinge for each electrode through which an electrode lead wire may pass.

15. The device of claim 13 further including at least one aperture on said biasing means for each electrode through which an electrode lead wire may pass.

16. The device of claim 14 wherein said biasing means is a living hinge.

17. The device of claim 15 wherein said first end includes a through aperture through which an electrode lead wire may pass.

18. The apparatuse of claim 16 further including one electrode storage tube for each electrode, said electrode storage tubes being held between said halves of said crimping tool for storing said electrodes.

19. The apparatus of claim 17 further including at least one notch in said jaws to accept each electrode.

20. The apparatus of claim 13 wherein said electrode holder further includes;
a groove in said cap in which said electrode rests.

21. A method of attaching a sutureless clip with an electrode releasably attached to a spring clip electrode holder to living tissue comprising the steps of:
a) inserting a sutureless clip with an electrode releasably attached to a spring clip electrode holder into a crimping tool;
b) placing a sutureless clip with an electrode releasably attached to a spring clip electrode holder in contact with living tissue;
c) crimping said sutureless clip with an electrode releasably attached to a spring clip electrode holder with said crimping tool to attach it to living tissue;
d) removing said sutureless clip with an electrode releasably attached to a spring clip electrode holder from said crimping tool.
